(19) United States
(12) Patent Application Publication
(10) Pub. No.: US 2013/0143320 A1
(43) Pub. Date: Jun. 6, 2013

(54) METHODS AND COMPOSITIONS FOR MANIPULATING THE GUIDED NAVIGATION OF ENDOTHELIAL TUBES DURING ANGIOGENESIS

(71) Applicants: Dean Y. Li, Salt Lake City, UT (US); Kye Won Park, Salt Lake City, UT (US)

(72) Inventors: Dean Y. Li, Salt Lake City, UT (US); Kye Won Park, Salt Lake City, UT (US)

(73) Assignee: University of Utah Research Foundation, Salt Lake City, UT (US)

(21) Appl. No.: 13/658,526

(22) Filed: Oct. 23, 2012

Related U.S. Application Data
(63) Continuation of application No. 12/975,160, filed on Dec. 21, 2010, now abandoned.
Application No. 12/975,160 is a continuation of application No. 10/519,342, filed on Sep. 21, 2005, now abandoned.
(60) Provisional application No. 60/392,142, filed on Jun. 27, 2002.

(51) Int. Cl. C12N 5/071; C07K 14/705

(52) U.S. Cl. CPC C12N 5/0691; C07K 14/705
USPC 435/375; 536/23.5; 530/350

(57) ABSTRACT

Methods and compositions for manipulating the directed navigation of physiological tracking tubular structures are provided. A novel cell-bound receptor, roundabout-4 (Robo-4), is described. The Robo-4 receptor shows sequence and structural similarity to members of the roundabout family of receptors. Also, the Robo-4 receptor binds Slit ligand, a known receptor of the roundabout receptors. Polynucleotides and polypeptides of the Robo-4 receptor are described.

### FIELD OF THE INVENTION

The present invention relates to methods and compositions that are useful in manipulating the guidance of physiological tracking tubular structures, such as endothelial tubes. In preferred embodiments, the invention relates to methods and compositions that are useful in manipulating the directed navigation of endothelial tubes, such as during angiogenesis, during embryonic development and in the neovascularization of tumors and other cell masses and/or tissues. More specifically, the present invention relates to a guidance system that can be used to both direct endothelial tubes toward a target, such as ischemic tissue, and direct tubes away from a target, such as a solid tumor.

### BACKGROUND OF THE INVENTION

The vasculature provides a network of vessels that efficiently delivers nutrients to and removes waste from tissues of the body. This network extends throughout most of the body, reaching all major tissues, and consists of two distinct types of structures—arteries and veins. The arterial and venous systems are parallel networks that function to deliver blood to a tissue and carry blood and waste away from tissues, respectively. These two systems are anatomically distinct and connect at distal capillary beds.

The network of vessels that comprise the arterial and venous systems develops by a process of directed movement of endothelial tubes to desired cell masses and/or tissues. During embryogenesis, the initial vascular framework is defined by the de novo formation of the dorsal aortae and cardinal veins. Mature circulatory networks are formed when endothelial tubes sprout from central vessels, navigate through the embryo, and reach their target cell mass and/or tissue. Upon reaching the target, the tubes are able to supply blood as nourishment for the tissue.

This navigation of endothelial tubes is important not only during embryonic development when the vasculature is first forming, but also in all physiological processes that include the introduction of a blood supply to a cell mass and/or tissue. These processes include various disease conditions that are sustainable only because of the introduction of a blood supply to a cell mass, such as the survival of a solid cancerous tumor. The continued growth of a solid tumor requires the presence of a blood supply that nourishes the cells of the tumor mass. Angiogenesis, a physiological process in which new blood vessels are formed and directed into a target cell mass and/or tissue, is a critical step in tumor development and survival. Accordingly, methods and compositions that are able to interfere with this process could be useful in preventing the growth and/or survival of tumors.

Other disease conditions exist in which the blood supply to a particular tissue is blocked or otherwise impeded, thereby diminishing the supply of nutrients to that particular tissue. For example, ischemia is a condition in which a localized anemia occurs in a tissue due to an obstruction of the inflow of arterial blood. This condition can be corrected by removal of the obstruction, or development of new vessels that are capable of supplying the required nourishment to the affected tissues.

Therefore, there is a need for compositions and methods that are able to manipulate the navigation of physiological tracking tubular structures, such as endothelial tubes, such that the structures can be directed towards a desired tissue, or prevented from reaching a target tissue.

### SUMMARY OF THE INVENTION

The present invention is directed at a guidance system and methods that function to direct navigation of physiological tubular structures, such as endothelial tubes. The invention includes a novel cell-bound receptor, Roundabout 4 (Robo-4), that is expressed in endothelial cells and interacts with a known ligand to affect directed navigation of endothelial tubes during vascular development. Together, the Robo-4 receptor and the ligand, the slit ligand, inhibit the directed navigation of endothelial tubes to target cell masses and/or tissues. Thus, the interaction between the Robo-4 receptor and the slit ligand provides a repulsive cue that affects the guidance of tubular structures, such as endothelial tubes. As described herein, the repulsive cue provided by Robo-4/slit interactions can be used to direct tubular structure both toward and away from a tissue or other cell mass of interest.

The present invention is useful for a variety of purposes. For example, the polynucleotides of the present invention can be used for gene therapy, such as replacement of defective copies of naturally occurring genes or provision of supplemental genes. Furthermore, the polypeptides of the present invention can be used in therapeutic procedures. For example, the polypeptide encoding the receptor, or a fragment thereof, can be supplied to an environment in order to compete with cell-bound receptors, thereby effectively lowering or preventing activation of the cell-bound receptors. Also, the various methods of the present invention are useful in studying and treating conditions related to angiogenesis, such as ischemia and tumor growth.

The inventors have identified and sequenced the gene that encodes the receptor, identified at least one ligand for the receptor (the slit ligand), and identified sequence and structural similarities between the novel receptor and a family of existing receptors—the Roundabout family of receptors.

Also, the inventors have identified a function for the Robo-4 receptor. The receptor, following interaction with the slit ligand, inhibits the migration of endothelial tubes. The repulsive cue provided by Robo-4/slit interaction contributes to the directed navigation of endothelial tubes by steering the tubes away from a location having the ligand, such as a cell expressing the ligand.

The Robo-4 receptor is expressed on sprouting endothelial tubes that form the perineural vasculature beds. The neural tubes produce and secrete the slit ligand. This enables the directed navigation of the endothelial tubes away from the neural tubes. As a result of this negative cue and likely in combination with attractive cues, the endothelial tubes, through slit-Robo-4 binding interactions that result in directed navigation, form a vasculature network around the developing central nervous system. This interaction with the central nervous system leads to the close association between the nervous and vasculature systems that is evident in both macro and micro anatomies.

Thus, the present invention includes the isolated cDNA and polypeptides of the Robo-4 receptor. Also, the invention includes methods of manipulating the guided navigation of endothelial tubes based on interactions between the Robo-4 receptor and the slit ligand. Further, the invention includes methods of inducing and preventing angiogenesis by inhibiting and activating, respectively, the Robo-4 receptor.

In a preferred embodiment, the method of the present invention comprises a method of directing the navigation of endothelial tubes away from a target by allowing binding between the slit ligand and the Robo-4 receptor on the endothelial cells of the tubes. The directed navigation of endothelial tubes away from target tissue in this method can be accomplished by expressing slit ligand in cells of the target tissue.

In a second preferred embodiment, the method of the present invention comprises a method of inducing the directed navigation of endothelial tubes toward a first target cell mass and/or tissue by repelling the endothelial cells away from a second target through Robo-4/slit binding. This can be accomplished by expressing the slit ligand in the second target and exposing the endothelial tubes to the second target. In a particularly preferred embodiment, a substantially continuous second target, such as a tissue surface or vessel, is lined with slit ligand, thereby providing a continuous repulsive force away from the second target.

### BRIEF DESCRIPTION OF THE DRAWINGS

FIG. 1 represents results of a Northern Blot analysis of Robo4 expression in Alk1+/+ and Alk1−/− tissues. The bottom panel shows loading controls, 28S and 18S RNA.

FIG. 2 represents visualization of staining of Robo4 anti-sense cRNA at days 9.0 and 9.5 of embryonic development.

FIG. 3 is a schematic comparing various domains of various members of the Robo family of receptors.

FIG. 4 is a schematic illustrating the phylogeny of some members of the Robo family of receptors.

FIG. 5 represents visualization of an immunoblotting assay in which Human Slit 2-myc was coimmunoprecipitated with Robe 4-HA using anti-HA antibodies.

FIGS. 6A-6F graphically represent data from various cell migration assays.

FIG. 7 graphically represents data from a cell migration assay utilizing human microvascular endothelial cells (HMVECs).

### DETAILED DESCRIPTION OF PREFERRED EMBODIMENTS OF THE INVENTION

The following description of preferred embodiments provides examples of the present invention. The embodiments discussed herein are merely exemplary in nature, and are not intended to limit the scope of the invention in any manner. Rather, the description of these preferred embodiments serves to enable a person of ordinary skill in the relevant art to make and use the present invention.

Activin receptor-like kinase 1 (Alk-1) is a receptor that plays a role in vascular development. Loss of function mutations in the Alk-1 receptor are responsible for Hereditary Hemorrhagic Telangiectasia (HHT), an autosomal dominant vascular dysplasia. (Johnson D W, Berg J N, Baldwin M A, Gallione C J, Marondel I, Yoon S J, Stenzel T T, Speer M, Perciak-Vance M A, Diamond A, Guttmacher A E, Jackson C E, Attisano L, Kucherlapati R, Porteous M E, Marchuk D A. (1996), Mutations in the activin receptor-like kinase 1 gene in hereditary hemorrhagic telangiectasia type 2. Nat Genet. 13(2):189-95; Berg J N, Gallione C J, Stenzel T T, Johnson D W, Allen W P, Schwartz C E, Jackson C E, Porteous M E, Marchuk D A. (1997), The activin-like kinase 1 gene: genomic structure and mutations in hereditary hemorrhagic telangiectasia type 2. Am J Hum Genet. 61 (1):60-7).

The inventors have generated and characterized genetic knockout mice that lack functional Alk-1. (Urness, L. D., Sorenson, L. K., Li, D. Y. (2000) Arteriousvenous malformations in mice lacking activin receptor-like kinase-1. Nature Genetics. 26:328-331). These mice are described in our U.S. patent application Ser. No. 09/578,553, which is hereby incorporated by reference in its entirety. In homozygous Alk-1−/− embryos, the distinct anatomical, structural, molecular, and functional properties of arteries and veins are lost. As a result, the development of these embryos is arrested at about day 10.5. Based on these studies, the inventors have discovered that Alk-1 regulates molecular programs that instruct sprouting arteries and veins to remain distinct as they are guided along parallel pathways to common distal target organs.

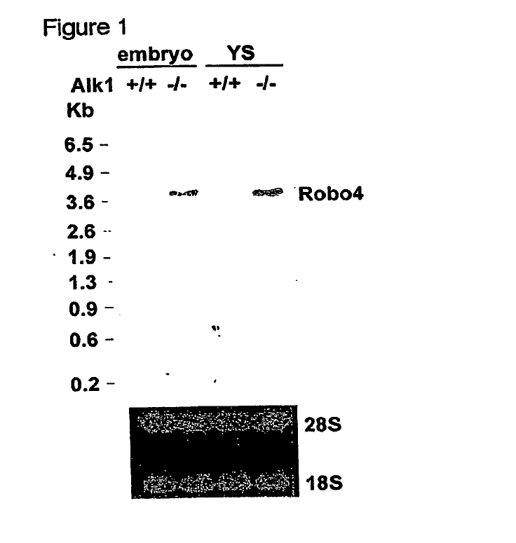

To further characterize the role of Alk-1 in vascular development, the inventors have investigated genes that are differentially expressed in Alk-1+/+ and Alk-1−/− cells. A screen of these differentially expressed genes revealed a novel receptor, which the inventors have termed the Roundabout 4 (Robo-4) receptor. These differential expression studies showed that Robo-4 is expressed in Alk-1−/− mice at levels that are approximately 4 to 5 fold higher than those in wild-type mice (See FIG. 1).

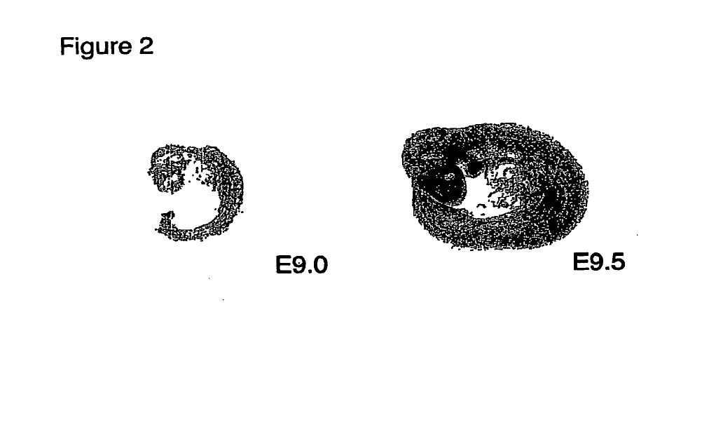

Also, in situ hybridization of Robo-4 shows the temporal and spatial expression of Robo-4 in vascular tissues (See FIG. 2). Between E8.0 to 8.5 Robo-4 was expressed in the central vessels, the dorsal aortae and cardinal veins. Between E8.5 and E10.0, intersomitic vessels sprout and a capillary plexus forms around the neural tube. Robo-4 expression was detected throughout the endothelium of these structures during this critical period of angiogenesis. In cross-sections, the expression of Robo-4 was more prominent in smaller vessels and capillary beds than in large vessels such as the dorsal aortae and cardinal veins. Robo-4 expression was detected in the endothelial cells that invaded the neural tube, but never in the neural tissue proper. This is in contrast with Robo-1, Robo-2, and Robo-3 which are highly expressed in the nervous system of mice, chick and zebrafish consistent with their roles in neuronal migration and axonal guidance (13-18). During zebrafish development, zfRobo4, with its unique extracellular domain structure of three IgG and two FN domains, was expressed in both the developing neural tube and vascular system. Northern blot analysis indicated that Robo-4 is expressed throughout embryogenesis and during adulthood. Robo-4 expression was highest in the heart and was undetectable within the brain, spleen, and testis. Interestingly, Robo-4 was expressed at intermediate levels in tracking tubular structures in the liver, kidney, and lung, such as bronchioles. Northern blot analysis for Robo-1 expression demonstrated prominent brain expression consistent with previously published reports (13). These results demonstrate that during development, Robo-4 differs from other Robe family member in its prominent endothelial expression pattern.

The inventors have cloned and sequenced both the human and mouse Robo-4 genes. The mouse Robo-4 cDNA sequence appears as SEQ ID 1, and the human Robo-4 cDNA appears as SEQ ID 2. Also, the deduced amino acid sequence for the mouse Robo-4 receptor appears as SEQ ID 3 and the deduced amino acid sequence for the human Robo-4 receptor appears as SEQ ID 4. The invention includes isolated polynucleotides that encode a Robo-4 receptor, complimentary polynucleotide sequences, and fragments and portions thereof, including the polynucleotides listed herein as SEQ ID 1 and SEQ ID 2 and complimentary nucleic acid molecules of these polynucleotides.

As used herein, the term “isolated” refers to a molecule that is purified from the setting in which it is found in nature and is separated from at least one contaminant molecule of the same class of molecules. Thus, an isolated polynucleotide comprises a polynucleotide that is purified from its natural setting and separated from at least one contaminant polynucleotide. Similarly, an isolated polypeptide comprises a polypeptide that is purified from its natural setting and separated from at least on contaminant polypeptide. As used herein, the term “complementary nucleic acid molecule” refers to a polynucleotide that is sufficiently complementary to a sequence, e.g., SEQ ID NOS 1 and 2, such that hydrogen bonds are formed with few mismatches, forming a stable duplex. As used herein, the term “complementary” refers to Watson-Crick or Hoogsteen base pairing between nucleotides.

The invention also includes derivative, analog, and homolog nucleic acid molecules of the polynucleotides of the invention, including the polynucleotides listed herein as SEQ ID 1 and SEQ ID 2. As used herein, the term “derivative nucleic acid molecule” refers to a nucleic acid sequences formed from native compounds either directly or by modification or partial substitution. As used herein, the term “analog nucleic acid molecule” refers to nucleic acid sequences that have a structure similar, but not identical, to the native compound but differ from it in respect to certain components or side chains. Analogs may be synthesized or from a different evolutionary origin. As used herein, the term “homolog nucleic acid molecule” refers to nucleic acid sequences of a particular gene that are derived from different species.

Derivatives and analogs may be full length or other than full length, if the derivative or analog contains a modified nucleic acid or amino acid. Derivatives or analogs of the polynucleotides of the invention include, but are not limited to, molecules comprising regions that are substantially homologous to the polynucleotides of the invention, including the polynucleotides listed herein as SEQ ID 1 and SEQ ID 2 by at least about 70%, 80%, or 95% identity over a nucleic acid of identical size or when compared to an aligned sequence in which the alignment is done by a homology algorithm, or whose encoding nucleic acid is capable of hybridizing to the complement of a sequence encoding a Robo-4 receptor.

“Homologous” nucleotide sequences encode those sequences coding for isoforms of the Robo-4 receptor. Homologous nucleotide sequences include nucleotide sequences encoding a polynucleotide for a Robo-4 receptor of species other than humans, such as vertebrates, e.g., frog, mouse, rat, rabbit, dog, cat, cow and horse. The polynucleotide listed herein as SEQ ID 1 is a cDNA sequence for the mouse Robo-4 receptor. Homologous nucleotide sequences also include naturally occurring allelic variations and mutations of the nucleotide sequences. A homologous nucleotide sequence does not, however, include the exact nucleotide sequence encoding the human Robo-4 receptor. Homologous nucleic acid sequences also include those nucleic acid sequences that encode conservative amino acid substitutions as well as a polypeptide possessing Robo-4 receptor biological activity. A conservative amino acid substitution is a change in the amino acid sequence that does not affect biological activity of the receptor.

In addition to the polynucleotide sequences shown in SEQ ID NOS 1 and 2, DNA sequence polymorphisms that change the amino acid sequences of the Robo-4 receptor may exist within a population. For example, allelic variation among individuals will exhibit genetic polymorphism in the Robo-4 receptor. As used herein, a “variant polynucleotide” is a nucleic acid molecule, or a complementary nucleic acid molecule, which encodes an active Robo-4 receptor that has at least about 80% nucleic acid sequence identity with a nucleic acid sequence encoding a full-length native Robo-4 receptor, or any other fragment of a full-length Robo-4 nucleic acid or complementary nucleic acid. Ordinarily, a variant polynucleotide will have at least about 80% nucleic acid sequence identity, more preferably at least about 81%, 82%, 83%, 84%, 85%, 86%, 87%, 88%, 89%, 90%, 91%, 92%, 93%, 94%, 95%, 96%, 97%, 98% nucleic acid sequence identity and yet more preferably at least about 99% nucleic acid sequence identity with a nucleic acid sequence encoding a full-length native Robo-4 receptor, or complimentary nucleic acid molecule. Variant polynucleotides do not encompass the native nucleotide sequence.

The invention also includes isolated polypeptides comprising a Robo-4 receptor, including the polypeptides having the amino acid sequences listed herein as SEQ ID 3 and SEQ ID 4.

The invention also includes derivative, analog, and homolog polypeptides of those listed herein as SEQ ID NOS 3, 4, 5, and 6. As used herein, the terms derivative amino acid sequence, analog amino acid sequence, and homolog amino acid sequence have the same meaning as for the nucleic acid terms, described above, applied to polypeptides.

As a preliminary matter, the inventors determined whether the identified genes share any sequence homology with any known families of receptors. Analysis of the Robo-4 gene sequences revealed significant homology with members of the Roundabout (Robo) family of receptors, which function in the guidance of neural tubes during development. The results of this analysis revealed that the Robo-4 gene shares 45% sequence similarity and 31% identity to members of the Robo family. Also, the inventors determined that the Robo-4 gene has a chromosomal position adjacent that of the Robo-3 gene, Rig-1. Accordingly, the inventors named the novel receptor Robo-4 due to this sequence homology as well as structural homology and functional similarities, as described below.

The human and mouse Robo-4 cDNA's encode proteins of 1007 and 1012 amino acids, respectively. The deduced polypeptide sequence includes a signal sequence of 20 amino acids and a single transmembrane domain. Further, structural analysis of the polypeptide sequence revealed the presence of two IgG domains as a well as two fibronectin domains. The IgG and fibronectin domains are all located to one side of the transmembrane domain. This arrangement is a structural feature shared by all members of the Robo family (See FIG. 3). Also, the Robo-4 polypeptide includes two cytoplasmic domains that are partially conserved (See FIG. 3 in which the partially conserved domains are labeled as domains 0 and 2).

FIG. 4 illustrates the phylogeny of the Robo family of receptors. In the Figure, the length of lines is proportional to the evolutionary distance between branch points. As the Figure shows, Robo-4 is closely associated with the Robo family of receptors.

Based on the observed sequence and structural similarities between the novel Robo-4 receptor and the Robo family of receptors, the inventors hypothesized that the Robo-4 receptor is a member of the Robo family. To confirm this hypothesis, the inventors evaluated the ability of the Robo-4 receptor to bind Slit2, a known ligand of receptors of the Robo family.

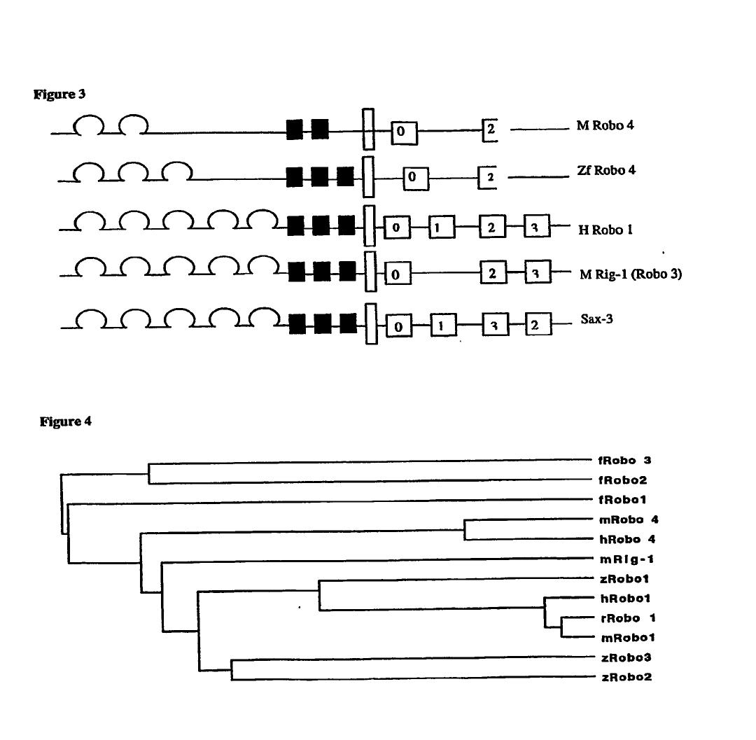

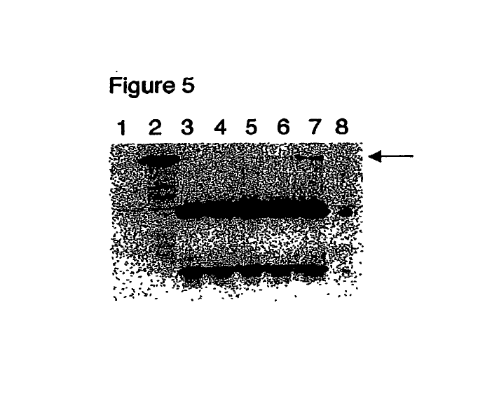

The Slit ligands show promiscuous binding to receptors of the Robo family. (Johnson D W, Berg J N, Baldwin Ma, Gallione C J, Marondel I, Yoon S J, Stenzel T T, Speer M, Perciak-Vance M A, Diamond A, Guttmacher A E, Jackson C E, Attisano L, Kucheerlapati R, Porteous M E, Marchuk D A. (1996), Mutations in the activin receptor-like kinase 1 gene hereditary hemorrhagic telangiectasia type 2. Nat Genet. 13(2):189-95; Berg J N, Gallione C J, Stenzel T T, Johnson D W, Allen W P, Schwartz C E, Jackson C E, Porteous M E, Marchuk D A. (1997), The activin-like kinase gene: genomic structure and mutations in hereditary hemorrhagic telangiectasia type 2. Am J Hum Genet. 61 (1):60-7; Urness, L. O., Sorenson, L. K., Li, D. Y. (2000), Arteriousvenous malformations in mice lacking activin receptor-like kinase-1. Nature Genetics. 26:328-331). The inventors investigated the ability of mouse Robo-4 receptor to bind human Slit2 ligand. For these experiments, stable cells line expressing full length hemagglutinin-tagged Robo-4 receptors (Robo-4-HA) were generated. These cells also expressed a secreted form of hemagglutinin-tagged Robo-4. All constructs were confirmed by sequencing and western blotting. Human Slit2 ligand tagged with the Myc Epitope was stably transfected into HEK 293 cells. Immunoprecipitation studies indicated that Robo-4-HA and Human Slit2-Myc complexes could be coprecipitated by antibodies against HA (See FIG. 5, particularly gel lane 7). This demonstrates that the Robo-4 receptor binds the Slit2 ligand.

The binding of Human Slit2 to the mouse Robo-4 receptor is saturable. Thus, the Robo-4 receptor specifically binds the Slit2 ligand, confirming the identity of the novel receptor as a member of the roundabout family of receptors (the Robo receptors). Immunoprecipitation data was confirmed by determining whether Slit protein bound to membranes of cells expressing Robo-4. HEK cells expressing Robo-4 (Robo-4-HEK) or Control-HEK cells were incubated with conditioned media from Slit-expressing cells (Slit-myc CM). Binding of Slit-myc proteins to the cell surfaces was detected by indirect immunofluoresence using a murine anti-myc antibody and an Alexa 594 conjugated anti-mouse antibody. Fluorescence was detected on the surface of Robo-4-HEK cells and not Control-HEK cells. Together, the immunoprecipitation and immunofluorescence data provide strong evidence that Slit binds to Robo-4 on the cell surface.

The Robo receptors have a well-defined function in neural guidance. (Song, H, Poo M. (2001), The cell biology of neuronal navigation. Nat Cell Biol (3):E81-8; Brose K, Tessier-Lavigne M. (2000), Slit proteins: key regulators of axon guidance, axonal branching, and cell migration. Curr Opin Neurobiol. 10(1):95-102; Wong K, Ren X R, Huang Y Z, Xie Y, Liu G, Saito H, Tang H, Wen L, Brady-Kalnay S M, Mei L, Wu J Y, Xiong W C, Rao Y. (2001), Signal transduction in neuoronal migration roles of gtpase activating proteinc and the small gtpase cdc42 in the slit-robo pathway. Cell, 107(2):209-21; Guthrie S. Axon guidance:Robos make the rules (2001), Curr Biol 17; 11(8):R300-3; Battye R, Stevens A, Jacobs J R. (1999), Axon repulsion from the midline of the Drosophila CNS requires slit function. Development. 126(11):2475-81; Li H S, Chen J H, Wu W, Fagaly T, Zhou L, Yuan W, Dupuis S. Jiang Z H, Nash W, Gick C, Omitz O M, Wu J Y, Rao Y. (1999), Vertebrate slit, a secreted ligand for the transmembrane protein roundabout, is a repellant for olfactory bulb axons. Cell 96(6):807-18; Brose K, Bland K S, Wang K H, Arnott D, Henzel W, Goodman C S, Tessier-Lavigne M, Kidd T. (1999), Slit proteins bind Robo receptors and have an evolutionary conserved role in repulsive axon guidance. Cell 96(6):795-806). In the neural system, a series of repulsive and attractive cues provide a guidance system for directing the navigation of axons to and/or away from targets. The Robo receptors, in conjunction with the Slit ligands, are critical for guiding axons to synapse with the appropriate distal targets through repulsive cues. Thus, in the neural system, the Slit ligands and some of the previously known Robo receptors direct the navigation of neurons during development of the neural system.

Considering the sequence and structural similarities between the novel Robo-4 receptor and the Robo family of receptors, and also the expression of the Robo-4 receptor in vascular cells, such as endothelial tubes, the inventors hypothesized that the novel receptor has a function in directing the navigation of the vasculature during its development by way of a repulsive cue. To investigate this proposed function, the inventors have evaluated the ability of the Slit ligand to affect behavior of endothelial tubes via interaction with the Robo-4 receptor.

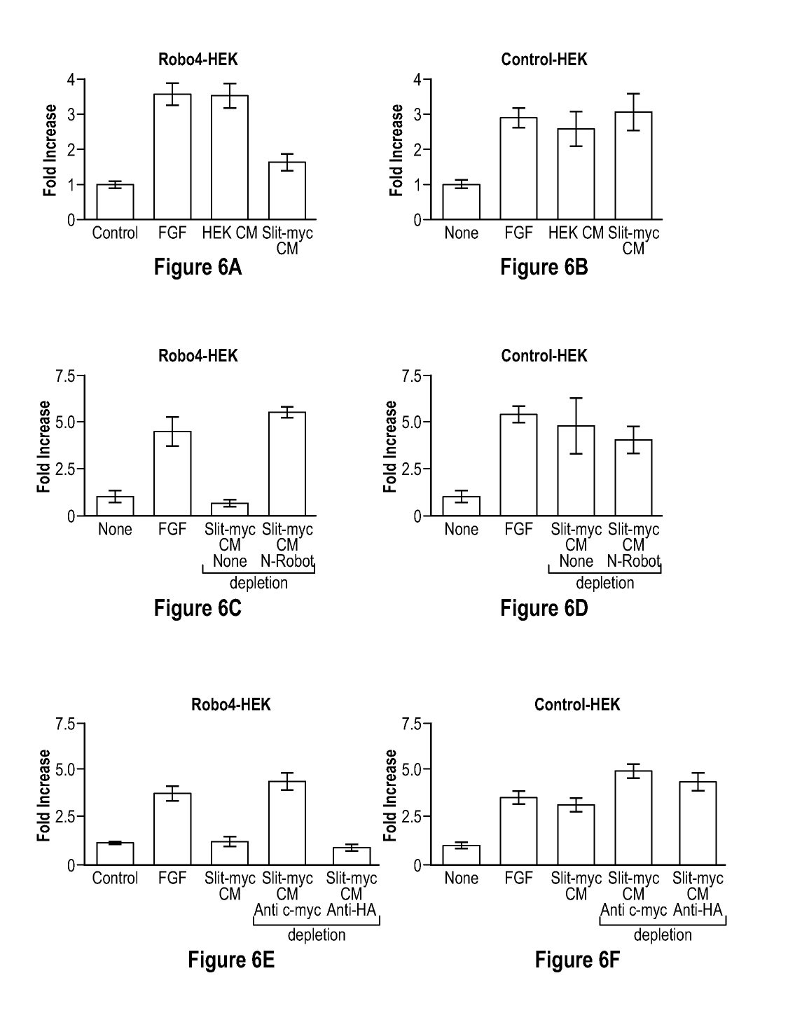

To confirm the function of the Robo-4 receptor observed in vitro, the inventors examined the function of the receptor in vivo. In addition to their role in neuronal guidance, it has recently been shown that Slit inhibits the migration of HEK cells that express Robo-1 (22). The inventors have found that Slit had a similar effect in cells expressing Robo-4. For these studies, standard transfilter assay were performed in which test factors were placed in the lower chamber and cells were placed in the upper chamber. The number of cells that migrated to the lower chamber after 2 hours was determined. In these experiments, the migration of Robo-4-HEK and Control-HEK cells to Slit-myc conditioned media (CM) as well as to media collected from control HEK cells, i.e., media lacking slit, was observed. Slit specifically inhibited the migration of Robo-4 expressing HEK cells. As expected, fibroblast growth factor and HEK-CM induced both Robo-4-HEK and Control-HEK cells to migrate at a rate of three to four-fold greater than background (FIG. 6A). Slit-myc CM induced comparable levels of migration of Control-HEK cells (FIG. 6B). However, when applied to Robo-4-HEK cells, Slit-myc CM inhibited migration to baseline levels (FIG. 6A).

This inhibitory effect of Slit-myc CM was specific for the Slit protein (FIG. 6C-6F). Conditioned media from HEK cells that expressed the soluble ligand binding ectodomain of Robo-1 (NRobo-1-HA) was incubated with Slit-myc CM. The binding of NRobo-1 to Slit-myc is an effective method for removing Slit protein from conditioned media (21-23). The inhibitory effect of Slit-myc CM on the migration of Robo-4-HEK cells was lost following depletion with NRobo-1 (FIG. 6C, 6D). Similarly, Slit-myc CM pretreated with an anti-myc antibody lost its inhibitory effect on Robo-4 HEK cell migration (FIG. 6E, 6F). Mock depletions with an anti-HA antibody did not reduce the inhibitory effect of Slit-myc CM.

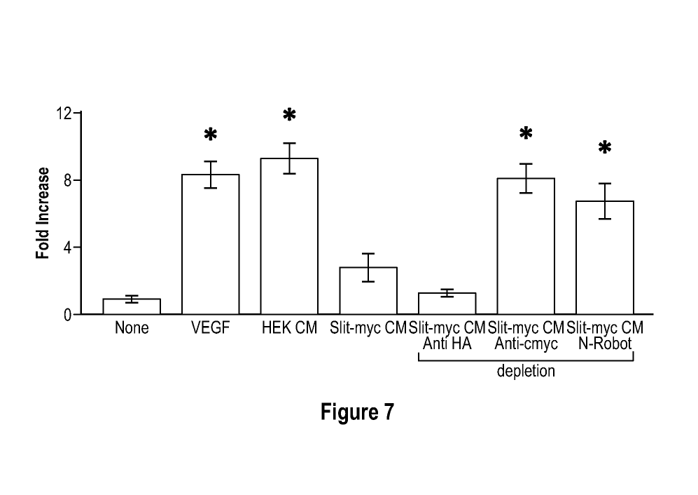

Slit modulates endothelial cell migration via Robo-4. To demonstrate that Robo-4 was present on the cell surface of primary endothelial cells, the inventors generated a polyclonal antibody to its cytoplasmic region (amino acids 964-981). This region is highly conserved between human and mice, and is specific to Robo-4. Culture media from HEK cells induced migration of human microvascular endothelial cells (HMVECs) at a level comparable to 10 ng/ml of vascular endothelial growth factor (VEGF) (FIG. 7). However, with Slit-myc CM, there was a 70% inhibition of migration (FIG. 7). Depleting Slit protein from Slit-myc CM with anti-myc antibody or NRobo-1 blocked in the inhibitory effect of Slit-myc CM on endothelial cell migration (FIG. 7). The inhibitory effect of Slit on Robo-4 expressing endothelial cells mirrored that of Robo-4-HEK cells (FIG. 7). Thus, Slit binds and activates Robo-4 in primary endothelial cells.

The function of the Robe receptor makes the receptor useful in a variety of methods relevant to medicine and research. Specifically, because the Robo-4 receptor provides a repulsive cue in the directed navigation of endothelial tubes during angiogenesis, the receptor and Slit ligand can be used to manipulate this process. Accordingly, the present invention also includes methods of manipulating the guided navigation of endothelial tubes during angiogenesis.

In one preferred embodiment, the invention includes methods of directing the navigation of physiological tubular structures toward a target tissue. This method is useful to encourage the directed navigation of developing vasculature to a target cell mass and/or tissue. The method can be used to provide new vasculature to a cell mass/tissue that is in need of a new system of nutrient supply and waste removal. For example, an ischemic tissue suffers from reduced oxygen supply due to poor blood flow to the tissue. By encouraging angiogenesis to an ischemic tissue, a new blood supply route can be created, effectively providing a new nutrient supply and waste removal system for the tissue, which can help to correct the condition.

Thus, in one particularly preferred embodiment, the invention comprises a method of directing endothelial tubes to a first target cell mass and/or tissue by repelling the endothelial tubes away from a second target via Robo-4 binding interactions with a ligand of the receptor, such as a Slit. The repelling away from the second target can direct the endothelial tubes toward the first target. Due to the presence of the receptor and the ligand, the endothelial tubes will navigate away from the second target, and toward the first target.

Angiogenesis may be induced by inhibiting Robo-4 activation in endothelium by inhibiting activation of the Robo-4 receptor. The absence of the negative cues provided by Robo-4 activation may induce angiogenesis in the tissue, which may be independent of directional limitations. The inhibition of activation of the Robo-4 receptor can be accomplished in any suitable manner, such as by providing a soluble form of the receptor to the endothelium tissue. The presence of soluble receptor may bind any ligand that is present, which may prevent ligand bind to and activation of the cell-bound receptor. SEQ ID 5 and SEQ ID 6 provide mouse and human soluble receptor forms, respectively. Also, fragments of these sequences may be suitable for use in the methods of the invention, as may sequences with less than 100% homology to these sequences. Particularly preferred sequences have 80% sequence identity to SEQ ID 6, or a fragment thereof.

In a second preferred embodiment, the present invention includes methods of preventing angiogenesis to a target by directing endothelial tubes away from the target. The presence of a blood supply is vital to survival of cell masses and/or tissues. In some instances, it may be desirable to remove the blood supply or prevent its formation and/or generation in order to lyse the cell mass and/or tissue by removing its nutrient supply. For example, cancerous cell masses, such as solid tumors, ensure their long-term survival by developing a blood supply through angiogenesis. By preventing this development, the methods of the present invention provide techniques for lysing cell masses and/or tissues.

Thus, in one particularly preferred embodiment, the invention includes methods of preventing the guided navigation of endothelial tubes during angiogenesis to a target cell mass/tissue. The method according to this embodiment includes exposing the endothelial tubes to a ligand of the Robo-4 receptor, such as the Slit ligand. The Slit ligand binds to the Robo-4 receptor on the endothelial tubes and inhibits their migration which interrupts the directed navigation of the endothelial tubes towards the cell mass and/or tissue. Any suitable technique for allowing binding between the receptor and ligand can be used. Preferred techniques include expressing the Slit ligand in the target and allowing the expressed Slit ligand to interact with the Robo-4 receptor on the endothelial tubes.

Angiogenesis may be inhibited and/or prevented generally, without a directional limitation, in endothelium by activating Robo-4 receptor in the tissue. Activation of the receptor can be accomplished by any suitable technique, such as by providing a ligand of the Robo-4 receptor to the receptor, and allowing the ligand to bind to the receptor. Slit ligand is a particularly preferred ligand. The ligand can be provided in any suitable manner, such as by providing a soluble form of the receptor directly to the endothelium, by expressing the ligand in cells of the endothelium or adjacent tissue, or other suitable techniques. Also, fragments of ligands of the Robo-4 receptor may be used. The fragment need only retain the ability to bind and activate the receptor. Also, activation of the Robo-4 receptor can be accomplished by other suitable techniques, such as by using agonosits of the Robo-4 receptor, including monoclonal and polyclonal antibodies that bind and activate the receptor.

In another preferred embodiment, the invention provides methods of disrupting the navigation of tracking tubular structures, such as endothelial tubes, that express the Robo-4 receptor. The negative cue provided by Slit/Robo-4 binding likely works in combination with positive cues that, together, provide a navigation system that directs tracking tubular structures toward and away from a series of local targets to ultimately direct the structures along a desired path. By interfering with the negative cue, the entire navigation system will be dysfunctional, and the tracking tubular structures will not be positioned on the desired path. This may result in the structures going in several directions, due to the presence of positive cues, but not in the path naturally desired due to the lack of counteracting negative cues. This can be useful in experimental work with and clinical treatment of conditions in which an excessive amount of tracking tubular structure penetration occurs. For example, in cancer, retinopathy, and inflammatory conditions, excessive neovasculartization occurs, and disruption of the navigation of endothelial tubes could interfere with this condition, which may ultimately limit the progression of the condition.

In a preferred embodiment, the methods of disrupting navigation comprise inhibiting activation of the Robo-4 receptor(s) of the tracking tubular structures. The inhibiting can be accomplished using various techniques suitable for accomplishing inhibition of activation of a cell-bound receptor, such as blocking the receptor with a monoclonal antibody or polyclonal immunoglobulin, or with other agents capable of specifically binding the receptor without activating the receptor. Also, a soluble receptor or receptor component can be prepared. The inventors have prepared a soluble form of the mouse Robo-4 receptor, termed N-Robo-4 and listed herein as SEQ ID 5. The N-Robo-4 composition contains the ectodomain (extracellular), but lacks the transmembrane and cytoplasmic domains of the cell-bound receptor. The amino acid sequence of the human N-Robo-4 composition is listed herein as SEQ ID 6. These soluble compositions will bind the ligand(s) of the receptor, such as Slit, and prevent their binding to and subsequent activation of the cell-bound receptor. These compositions may be engineered to include portions that enhance the effectiveness of the composition. For example, an immunoglobulin Fc segment can be added to the composition, which can facilitate removal of complexes of the composition and ligand through cells bearing Fc receptors, such as macrophages.

Other compositions capable of binding the ligand(s) of the receptor, such as Slit, could also be prepared and used to prevent ligand binding to the receptor. Examples of suitable such compositions include polyclonal and monoclonal antibodies capable of binding ligand(s). Further examples include soluble forms of other receptors capable of binding the Slit ligand, such as other Robo receptors.

The references cited in this disclosure are hereby incorporated into the disclosure in their entirety, except to any extent to which they contradict any statement or definition made herein.

The foregoing disclosure includes the best mode devised by the inventors for practicing the invention. It is apparent, however, that several variations may be conceivable by one skilled in the art. Inasmuch as the foregoing disclosure is intended to enable such person to practice the instant invention, it should not be construed to be limited thereby, but should be construed to include such aforementioned variations.

1. An isolated polynucleotide comprising SEQ ID 1.

2. An isolated polynucleotide comprising SEQ ID 2.

3. An isolated polypeptide comprising SEQ ID 3.

4. An isolated polypeptide comprising SEQ ID 4.

5. An isolated polypeptide comprising SEQ ID 5.

6. An isolated polypeptide comprising SEQ ID 6.

7. A method of directing the navigation of physiological tracking tubular structures that express Robo-4 receptor away from a target cell mass, comprising expressing a ligand of said Robo-4 receptor in said target cell mass and allowing binding between the ligand and said Robo-4 receptor.

8. The method of claim 7, wherein the ligand comprises Slit ligand.

9. The method of claim 7, wherein said physiological tracking tubular structures comprise endothelial tubes.

10. A method of directing the navigation of physiological tracking tubular structures that express Robo-4 receptor toward a target cell mass, comprising expressing a ligand of said Robo-4 receptor in a second cell mass and allowing binding between the ligand and said Robo-4 receptor.

11. The method of claim 10, wherein the ligand comprises Slit ligand

12. The method of claim 10, wherein said physiological tracking tubular structures comprise endothelial tubes.

13. A method of disrupting navigation of physiological tracking tubular structures that express Robo-4 receptor, comprising inhibiting activation of said Robo-4 receptor.

14. The method of claim 13, wherein said physiological tracking tubular structures comprise endothelial tubes.

15. A method of inducing angiogenesis in endothelium tissue expressing Robo-4 receptor, comprising inhibiting activation of said Robo-4 receptor.

16. The method of claim 15, wherein inhibiting activation of said Robo-4 receptor comprises providing a soluble form of said Robo-4 receptor to said endothelium tissue.

17. The method of claim 16, wherein the soluble form of said Robo-4 receptor comprises SEQ ID 6.

18. The method of claim 16, wherein the soluble form of said Robo-4 receptor comprises an amino acid sequence having at least 80% sequence identity to SEQ ID 6, or a fragment thereof.

19. A method of preventing angiogenesis in endothelium tissue expressing Robo-4 receptor, comprising activating said Robo-4 receptor.

20. The method of claim 19, wherein activating said Robo-4 receptor comprises providing a ligand of said Robo-4 receptor and allowing the ligand to bind to said Robo-4 receptor.

21. The method of claim 20, wherein the ligand comprises Slit ligand.

22. The method according to any of claim 7, 10 and 20, wherein the ligand comprises human Slit2 ligand, or a fragment thereof.


SEQUENCE LISTING (6 sequences)

SEQ ID NO: 1 - DNA, 3742 bp, Mus musculus
caaaagtgtatgggacaaggagaggagccgagagcagccatgggctctggaggaacgggcctcctggggacggagtggcctctgcctctgctgctgcttttcatcatgggaggtgaggctctggattctccaccccagatcctagttcacccccaggaccagctacttcagggctctggcccagccaagatgaggtgcagatcatccggccaaccacctcccactatccgctggctgctgaatgggcagcccctcagcatggccaccccagacctacattaccttttgccggatgggaccctcctgttacatcggccctctgtccagggacggccacaagatgaccagaacatcctctcagcaatcctgggtgtctacacatgtgaggccagcaaccggctgggcacagcagtgagccggggtgctaggctgtctgtggctgtcctccaggaggacttccagatccaacctcgggacacagtggccgtggtgggagagagcttggttcttgagtgtggtcctccctggggctacccaaaaccctcggtctcatggtggaaagacgggaaacccctggtcctccagccagggaggcgcacagtatctggggattccctgatggtgtcaagagcagagaagaatgactcggggacctatatgtgtatggccaccaacaatgctgggcaacgggagagccgagcagccagggtgtctatccaggaatcccaggaccacaaggaacatctagagcttctggctgttcgcattcagctggaaaatgtgaccctgctaaaccccgaacctgtaaaaggtcccaagcctgggccatccgtgtggctcagctggaaggtgagcggccctgctgcacctgctgagtcatacacagctctgttcaggactcagaggtcccccagggaccaaggatctccatggacagaggtgctgctgcgtggcttgcagagtgcaaagcttgggggtctccactggggccaagactatgaattcaaagtgagaccgtcctccggccgggctcgaggccctgacagcaatgtgttgctcctgaggctgcctgaacaggtgcccagtgccccacctcaaggagtgaccttaagatctggcaacggtagtgtctttgtgagttgggctccaccacctgctgaaagccataatggtgtcatccgtggttaccaggtctggagcctgggcaatgcctcattgcctgctgccaactggaccgtagtgggtgaacagacccagctggagatcgccacacgactgccaggctcctattgtgtgcaagtggctgcagtcactggagctggtgctggagaactcagtacccctgtctgcctccttttagagcaggccatggagcaatcagcacgagaccccaggaaacatgttccctggaccctggaacagctgagggccaccttgagacgaccagaagtcattgccagtagtgctgtcctactctggttgctgctactaggcattactgtgtgtatctacagacgacgcaaagctggggtgcacctgggcccaggtctgtacagatacaccagcgaggacgccattctaaaacacaggatggaccacagtgactccccatggctggcagacacctggcgttccacctctggctctcgagacctgagcagcagcagcagccttagtagtcggctgggattggaccctcgggacccactagagggcaggcgctccttgatctcctgggaccctcggagccccggtgtacccctgcttccagacacgagcacgttttacggctccctcattgcagagcagccttccagccctccagtccggccaagccccaagacaccagctgctaggcgctttccatccaagttggctggaacctccagcccctgggctagctcagatagtctctgcagccgcaggggactctgttccccacgcatgtctctgacccctacagaggcttggaaggccaaaaagaagcaggaattgcaccaagctaacagctccccactgctccggggcagccaccccatggaaatctgggcctgggagttgggaagcagagcctccaagaacctttctcaaagcccaggtccaaactctggttccccaggagaagcgccccgagccgtggtatcctggcgtgctgtgggaccacaacttcaccgcaactccagtgagctggcatctcgtccactccctccaacacccctttctcttcgtggagcttccagtcatgacccacagagccagtgtgtggagaagctccaagctccctcctctgacccactgccagcagcccctctctccgtcctcaactcttccagaccttccagcccccaggcctctttcctctcctgtcctagcccatcctccagcaacctgtccagctcctcgctgtcatccttagaggaggaggaggatcaggacagcgtgctcacccccgaggaggtagccctgtgtctggagctcagtgatggggaggagacacccacgaacagtgtatctcctatgccaagagctccttccccgccaacaacctatggctatatcagcataccaacctgctcaggactggcagacatgggcagagctggcgggggcgtggggtctgaggttgggaacttactgtatccacctcggccctgccccacccctacacccagcgagggctccctggccaatggttggggctcagcttctgaggacaatgtccccagcgccagggccagcctggttagctcttctgatggctccttcctcgctgatactcactttgctcgtgccctggcagtggctgtggatagctttggcctcagtctggatcccagggaagctgactgtgtcttcactgatgcctcatcacctccctcccctcggggtgatctctccctgacccgaagcttctctctgcctttgtgggagtggaggccagactggttggaagatgctgagatcagccacacccagaggctggggagggggctgcctccctggcctcctgattctagggcctcttcccagcgaagttggctaactggtgctgtgcccaaggctggtgattcctcctgaattgtccctgagaaggccagaagagcacccagaccactctcctgtctgtcccctggctttctcacatgtggaggtcttggcctatgcttctctgtaatagaagtccaccgtcactaggcttctggagagctctgtcattgggattgttaaaataaatgaaagcaaaccaaaatatgatcacgggagtcttggattcccactgagaacaagacagcatcttcaggacagcagactctccacaaccagaacctttggcctaagtaagcctggctccggagctcccacctaagtggatcatggaaagaagggaagccaaccaggtcttcaggaaggacagaaatgttttttggtgagggctatggtggaggacctgtggaagagccctctcatatctacttggactcctcccttagaggccagctcaaccctttccccagtcacaccatgcaaggaaactaaaggagaaaggtcgtggatgcagtgggccctatacagcgtcacagtcaatgcttcaaagtgagatcaatggaggagactgaaggaaaggacgcagggaaacagggaaccaatgcgctattctcattctaccgccactctgagcttaaggaacttaattctataaaactgtaaagacgaaaaaaaaaaaaaaaaaaaaaaaaaaaaa

SEQ ID NO: 2 - DNA, 3778 bp, Homo sapiens
gctgagagtagccatgggctctggaggagacagcctcctggggggcaggggttccctgcctctgctgctcctgctcatcatgggaggcatggctcaggactccccgccccagatcctagtccacccccaggaccagctgttccagggccctggccctgccaggatgagctgccaagcctcaggccagccacctcccaccatccgctggttgctgaatgggcagcccctgagcatggtgcccccagacccacaccacctcctgcctgatgggacccttctgctgctacagccccctgcccggggacatgcccacgatggccaggccctgtccacagacctgggtgtctacacatgtgaggccagcaaccggcttggcacggcagtcagcagaggcgctcggctgtctgtggctgtcctccgggaggatttccagatccagcctcgggacatggtggctgtggtgggtgagcagtttactctggaatgtgggccgccctggggccacccagagcccacagtctcatggtggaaagatgggaaacccctggccctccagcccggaaggcacacagtgtccggggggtccctgctgatggcaagagcagagaagagtgacgaagggacctacatgtgtgtggccaccaacagcgcaggacatagggagagccgcgcagcccgggtttccatccaggagccccaggactacacggagcctgtggagcttctggctgtgcgaattcagctggaaaatgtgacactgctgaacccggatcctgcagagggccccaagcctagaccggcggtgtggctcagctggaaggtcagtggccctgctgcgcctgcccaatcttacacggccttgttcaggacccagactgccccgggaggccagggagctccgtgggcagaggagctgctggccggctggcagagcgcagagcttggaggcctccactggggccaagactacgagttcaaagtgagaccatcctctggccgggctcgaggccctgacagcaacgtgctgctcctgaggctgccggaaaaagtgcccagtgccccacctcaggaagtgactctaaagcctggcaatggcactgtctttgtgagctgggtcccaccacctgctgaaaaccacaatggcatcatccgtggctaccaggtctggagcctgggcaacacatcactgccaccagccaactggactgtagttggtgagcagacccagctggaaatcgccacccatatgccaggctcctactgcgtgcaagtggctgcagtcactggtgctggagctggggagcccagtagacctgtctgcctccttttagagcaggccatggagcgagccacccaagaacccagtgagcatggtccctggaccctggagcagctgagggctaccttgaagcggcctgaggtcattgccacctgcggtgttgcactctggctgctgcttctgggcaccgccgtgtgtatccaccgccggcgccgagctagggtgcacctgggcccaggtctgtacagatataccagtgaggatgccatcctaaaacacaggatggatcacagtgactcccagtggttggcagacacttggcgttccacctctggctctcgggacctgagcagcagcagcagcctcagcagtcggctgggggcggatgcccgggacccactagactgtcgtcgctccttgctctcctgggactcccgaagccccggcgtgcccctgcttccagacaccagcactttttatggctccctcatcgctgagctgccctccagtaccccagccaggccaagtccccaggtcccagctgtcaggcgcctcccaccccagctggcccagctctccagcccctgttccagctcagacagcctctgcagccgcaggggactctcttctccccgcttgtctctggcccctgcagaggcttggaaggccaaaaagaagcaggagctgcagcatgccaacagttccccactgctccggggcagccactccttggagctccgggcctgtgagttaggaaatagaggttccaagaacctttcccaaagcccaggagctgtgccccaagctctggttgcctggcgggccctgggaccgaaactcctcagctcctcaaatgagctggttactcgtcatctccctccagcacccctctttcctcatgaaactcccccaactcagagtcaacagacccagcctccggtggcaccacaggctccctcctccatcctgctgccagcagcccccatccccatccttagcccctgcagtccccctagcccccaggcctcttccctctctggccccagcccagcttccagtcgcctgtccagctcctcactgtcatccctgggggaggatcaagacagcgtgctgacccctgaggaggtagccctgtgcttggaactcagtgagggtgaggagactcccaggaacagcgtctctcccatgccaagggctccttcaccccccaccacctatgggtacatcagcgtcccaacagcctcagagttcacggacatgggcaggactggaggaggggtggggcccaaggggggagtcttgctgtgcccacctcggccctgcctcacccccacccccagcgagggctccttagccaatggttggggctcagcctctgaggacaatgccgccagcgccagagccagccttgtcagctcctccgatggctccttcctcgctgatgctcactttgcccgggccctggcagtggctgtggatagctttggtttcggtctagagcccagggaggcagactgcgtcttcatagatgcctcatcacctccctccccacgggatgagatcttcctgacccccaacctctccctgcccctgtgggagtggaggccagactggttggaagacatggaggtcagccacacccagcggctgggaagggggatgcctccctggccccctgactctcagatctcttcccagagaagtcagctccactgtcgtatgcccaaggctggtgcttctcctgtagattactcctgaaccgtgtccctgagacttcccagacgggaatcagaaccacttctcctgtccacccacaagacctgggctgtggtgtgtgggtcttggcctgtgtttctctgcagctggggtccaccttcccaagcctccagagagttctccctccacgattgtgaaaacaaatgaaaacaaaattagagcaaagctgacctggagccctcagggagcaaaacatcatctccacctgactcctagccactgctttctcctctgtgccatccactcccaccaccaggttgttttggcctgaggagcagccctgcctgctgctcttcccccaccatttggatcacaggaagtggaggagccagaggtgcctttgtggaggacagcagtggctgctgggagagggctgtggaggaaggagcttctcggagccccctctcagccttacctgggcccctcctctagagaagagctcaactctctcccaacctcaccatggaaagaaaataattatgaatgccactgaggcactgaggccctacctcatgccaaacaaagggttcaaggctgggtctagcgaggatgctgaaggaagggaggtatgagaccgtaggtcaaaagcaccatcctcgtactgttgtcactatgagcttaagaaatttgataccataaaatggtaaagacttgaaaaaaaaaaaaaaaaaaaaaaaaaaaaaaaaaaaaaaaaaaaaaaaaaaaaaaaaaaaaaaaaaaaaa

SEQ ID NO: 3 - protein, 1012 aa, Mus musculus
MGSGGTGLLGTEWPLPLLLLFIMGGEALDSPPQILVHPQDQLLQGSGPAKMRCRSSGQPPPTIRWLLNGQPLSMATPDLHYLLPDGTLLLHRPSVQGRPQDDQNILSAILGVYTCEASNRLGTAVSRGARLSVAVLQEDFQIQPRDTVAVVGESLVLECGPPWGYPKPSVSWWKDGKPLVLQPGRRTVSGDSLMVSRAEKNDSGTYMCMATNNAGQRESRAARVSIQESQDHKEHLELLAVRIQLENVTLLNPEPVKGPKPGPSVWLSWKVSGPAAPAESYTALFRTQRSPRDQGSPWTEVLLRGLQSAKLGGLHWGQDYEFKVRPSSGRARGPDSNVLLLRLPEQVPSAPPQGVTLRSGNGSVFVSWAPPPAESHNGVIRGYQVWSLGNASLPAANWTVVGEQTQLEIATRLPGSYCVQVAAVTGAGAGELSTPVCLLLEQAMEQSARDPRKHVPWTLEQLRATLRRPEVIASSAVLLWLLLLGITVCIYRRRKAGVHLGPGLYRYTSEDAILKHRMDHSDSPWLADTWRSTSGSRDLSSSSSLSSRLGLDPRDPLEGRRSLISWDPRSPGVPLLPDTSTFYGSLIAEQPSSPPVRPSPKTPAARRFPSKLAGTSSPWASSDSLCSRRGLCSPRMSLTPTEAWKAKKKQELHQANSSPLLRGSHPMEIWAWELGSRASKNLSQSPGPNSGSPGEAPRAVVSWRAVGPQLHRNSSELASRPLPPTPLSLRGASSHDPQSQCVEKLQAPSSDPLPAAPLSVLNSSRPSSPQASFLSCPSPSSSNLSSSSLSSLEEEEDQDSVLTPEEVALCLELSDGEETPTNSVSPMPRAPSPPTTYGYISIPTCSGLADMGRAGGGVGSEVGNLLYPPRPCPTPTPSEGSLANGWGSASEDNVPSARASLVSSSDGSFLADTHFARALAVAVDSFGLSLDPREADCVFTDASSPPSPRGDLSLTRSFSLPLWEWRPDWLEDAEISHTQRLGRGLPPWPPDSRASSQRSWLTGAVPKAGDSS

SEQ ID NO: 4 - protein, 1007 aa, Homo sapiens
MGSGGDSLLGGRGSLPLLLLLIMGGMAQDSPPQILVHPQDQLFQGPGPARMSCQASGQPPPTIRWLLNGQPLSMVPPDPHHLLPDGTLLLLQPPARGHAHDGQALSTDLGVYTCEASNRLGTAVSRGARLSVAVLREDFQIQPRDMVAVVGEQFTLECGPPWGHPEPTVSWWKDGKPLALQPGRHTVSGGSLLMARAEKSDEGTYMCVATNSAGHRESRAARVSIQEPQDYTEPVELLAVRIQLENVTLLNPDPAEGPKPRPAVWLSWKVSGPAAPAQSYTALFRTQTAPGGQGAPWAEELLAGWQSAELGGLHWGQDYEFKVRPSSGRARGPDSNVLLLRLPEKVPSAPPQEVTLKPGNGTVFVSWVPPPAENHNGIIRGYQVWSLGNTSLPPANWTVVGEQTQLEIATHMPGSYCVQVAAVTGAGAGEPSRPVCLLLEQAMERATQEPSEHGPWTLEQLRATLKRPEVIATCGVALWLLLLGTAVCIHRRRRARVHLGPGLYRYTSEDAILKHRMDHSDSQWLADTWRSTSGSRDLSSSSSLSSRLGADARDPLDCRRSLLSWDSRSPGVPLLPDTSTFYGSLIAELPSSTPARPSPQVPAVRRLPPQLAQLSSPCSSSDSLCSRRGLSSPRLSLAPAEAWKAKKKQELQHANSSPLLRGSHSLELRACELGNRGSKNLSQSPGAVPQALVAWRALGPKLLSSSNELVTRHLPPAPLFPHETPPTQSQQTQPPVAPQAPSSILLPAAPIPILSPCSPPSPQASSLSGPSPASSRLSSSSLSSLGEDQDSVLTPEEVALCLELSEGEETPRNSVSPMPRAPSPPTTYGYISVPTASEFTDMGRTGGGVGPKGGVLLCPPRPCLTPTPSEGSLANGWGSASEDNAASARASLVSSSDGSFLADAHFARALAVAVDSFGFGLEPREADCVFIDASSPPSPRDEIFLTPNLSLPLWEWRPDWLEDMEVSHTQRLGRGMPPWPPDSQISSQRSQLHCRMPKAGASPVDYS

SEQ ID NO: 5 - protein, 470 aa, Mus musculus
MGSGGTGLLGTEWPLPLLLLFIMGGEALDSPPQILVHPQDQLLQGSGPAKMRCRSSGQPPPTIRWLLNGQPLSMATPDLHYLLPDGTLLLHRPSVQGRPQDDQNILSAILGVYTCEASNRLGTAVSRGARLSVAVLQEDFQIQPRDTVAVVGESLVLECGPPWGYPKPSVSWWKDGKPLVLQPGRRTVSGDSLMVSRAEKNDSGTYMCMATNNAGQRESRAARVSIQESQDHKEHLELLAVRIQLENVTLLNPEPVKGPKPGPSVWLSWKVSGPAAPAESYTALFRTQRSPRDQGSPWTEVLLRGLQSAKLGGLHWGQDYEFKVRPSSGRARGPDSNVLLLRLPEQVPSAPPQGVTLRSGNGSVFVSWAPPPAESHNGVIRGYQVWSLGNASLPAANWTVVGEQTQLEIATRLPGSYCVQVAAVTGAGAGELSTPVCLLLEQAMEQSARDPRKHVPWTLEQLRATLRRPE

SEQ ID NO: 6 - protein, 469 aa, Homo sapiens
MGSGGDSLLGGRGSLPLLLLLIMGGMAQDSPPQILVHPQDQLFQGPGPARMSCQASGQPPPTIRWLLNGQPLSMVPPDPHHLLPDGTLLLLQPPARGHAHDGQALSTDLGVYTCEASNRLGTAVSRGARLSVAVLREDFQIQPRDMVAVVGEQFTLECGPPWGHPEPTVSWWKDGKPLALQPGRHTVSGGSLLMARAEKSDEGTYMCVATNSAGHRESRAARVSIQEPQDYTEPVELLAVRIQLENVTLLNPDPAEGPKPRPAVWLSWKVSGPAAPAQSYTALFRTQTAPGGQGAPWAEELLAGWQSAELGGLHWGQDYEFKVRPSSGRARGPDSNVLLLRLPEKVPSAPPQEVTLKPGNGTVFVSWVPPPAENHNGIIRGYQVWSLGNTSLPPANWTVVGEQTQLEIATHMPGSYCVQVAAVTGAGAGEPSRPVCLLLEQAMERATQEPSEHGPWTLEQLRATLKRPE